(19) United States
(12) Patent Application Publication
(10) Pub. No.: US 2025/0032707 A1
(43) Pub. Date: Jan. 30, 2025

(54) SYSTEM AND METHOD FOR CONTROLLING A TARGET-CONTROLLED INFUSION BY SPECIFYING AN INDUCTION DOSE

(71) Applicant: Fresenius Vial SAS, Brézins (FR)

(72) Inventors: Christine Durand, Chassieu (FR); Alexandre Guerrini, Fontaine (FR)

(21) Appl. No.: 18/705,450

(22) PCT Filed: Oct. 24, 2022
(86) PCT No.: PCT/EP2022/079499

§ 371 Date: Apr. 26, 2024

(30) Foreign Application Priority Data
Oct. 29, 2021 (EP) 21315229.1

(51) Int. Cl. A61M 5/168; G16H 20/17; G16H 40/63

(52) U.S. Cl. CPC A61M 5/1684; G16H 20/17; G16H 40/63

(57) ABSTRACT

A system for controlling a target-controlled infusion for administering a drug to a patient (P), comprises at least one infusion device (31-33) for administering a drug to the patient (P), and a control device (2) which is configured to control operation of the at least one infusion device (31-33) in order to establish a drug concentration within the patient (P) based on a target concentration (CT), wherein the control device (2) is configured to execute a target-controlled infusion protocol using a mathematical model modelling a drug distribution in the patient's body for controlling operation of the at least one infusion device (31-33). The control device (2) is configured to calculate, based on a user-specified induction dose (I), a value of a drug concentration within the patient (P) using said mathematical model and to consider said value of the drug concentration at the start of execution of said target-controlled infusion protocol.

The invention relates to a system for controlling a target-controlled infusion for administering a drug to a patient according to the preamble of claim 1 and to a method for controlling a target-controlled infusion for administering a drug to a patient.

A system of this kind comprises at least one infusion device for administering a drug to the patient and a control device configured to control operation of the at least one infusion device. The control as performed by the control device herein is such that a drug concentration e.g. at an effect site within the patient is established which is at or at least close to a target concentration, wherein the target concentration may be constant over a period of time, or may vary such that a concentration within the patient is controlled to follow a certain concentration curve. Herein, the control device is configured to execute a target-controlled infusion protocol using a mathematical model modeling a drug distribution in the patient's body for controlling operation of the at least one infusion device.

“Target-controlled infusion” (TCI) generally refers to an infusion operation performed by an computer-assisted infusion system which calculates a substance concentration in a particular body compartment on the basis of a mathematical model and which, after setting a target concentration, adjusts the infusion rate such that the concentration in the body compartment of the patient converges towards and is kept at the predefined target concentration. TCI infusion systems generally consist of one or multiple infusion devices and a control device, which may be separate to the infusion devices or may be integrated into an infusion device.

For setting up an infusion operation, herein, patient specific parameters such as the patient's age, weight, gender, and drug specific parameters such as the type of drug, e.g. the type of anesthetic, and a desired target concentration in a body compartment of the patient, for example relating to a drug level in the patient's brain within an anesthesia procedure, may be entered into the system using a human machine interface. In addition, a suitable mathematical model, such as a pharmacokinetic/pharmacodynamic model out of a multiplicity of models defined in the system, may be selected for executing a target-controlled infusion protocol. When performing a target-controlled infusion operation, then, the control device executes the target-controlled infusion protocol and in this context calculates infusion rates in order to control one or multiple infusion devices for administering one or multiple specified drugs to the patient.

On the basis of an empirically determined population-pharmacokinetic model and using a known pharmacokinetic and patient-specific pharmacodynamic parameter set of a medicament (for example propofol) as well as by means of patient-specific data, a TCI system models a drug distribution (over time) within the patient's body by calculating drug concentrations in body compartments as defined within the model. During the execution of a target-controlled infusion protocol, herein, the mathematical model may be repeatedly adjusted according to measurement values relating to a drug concentration within the patient, for example by measuring a drug concentration in a patient's breath or in the patient's plasma (blood) compartment, or by measuring biological signals such as EEG or ECG signals or by deriving indices such as the so-called bi-spectral (BIS) index. According to measurement values the mathematical model is used during operation such that it suitably reflects the concentrations in the patient's body compartments according to the measurement values, such that patient-individual effects such as a patient-specific metabolism may be taken into account. The mathematical model may hence accurately model the drug concentration within the body, which may be used to control the infusion operation using one or multiple infusion devices in order to set or maintain a desired concentration e.g. in a desired effect site compartment within the patient to obtain a desired medical effect, such as an anesthetic effect during an anesthesia procedure.

Systems and methods for performing target-controlled infusion operations, in particular anesthetic operations, are for example known from EP 1 418 976 B1, WO 2016/160321 A1, and WO 2017/190966 A1.

Within a target-controlled infusion operation typically infusion is carried out automatically, wherein the control is such that the drug concentration within the patient, for example at an effect site or at a plasma site of the patient, converges towards a target concentration and is maintained at the target concentration. For programming such a target-controlled infusion operation, herein, a physician typically is required to enter a desired target concentration, such that a suitable control can be carried out for establishing a drug concentration according to the target concentration.

However, physicians typically are most familiar with manual infusion operations, for example in the context of an anesthesia operation, in which a physician manually defines a dose to be administered to a patient, the dose being chosen such that a desired effect according to the physician's experience is obtained. Hence, a physician is most familiar with defining a required dose, but may not be very familiar with defining a target concentration according to which a drug concentration should be established in order to reach a desired effect.

There hence is a desire to enable a physician to intuitively initiate a drug administration operation, in particular an anesthesia operation, making use of an automatic control by employing a target-controlled infusion protocol.

It is an object of the instant invention to provide a system and a method which allow for a reliable, yet intuitive control by employing a target-controlled infusion protocol, but at the same time taking into account user-specified information.

This object is achieved by means of a system comprising the features of claim 1.

Accordingly, the control device is configured to calculate, based on a user-specified induction dose, a value of a drug concentration within the patient using said mathematical model and to consider said value of the drug concentration at the start of execution of said target-controlled infusion protocol.

Within the system one or multiple infusion devices are controlled to administer a drug or multiple different drugs to a patient. The control device herein shall execute a target-controlled infusion protocol in order to establish a drug concentration within the patient based on a target concentration. Hence, by means of the control device an automatic control of an infusion operation shall take place in that a drug concentration for example at an effect site or at a plasma site within the patient is established which is at or close to a desired target concentration.

However, the control device in addition takes into account a user-specified induction dose which has been administered already previously or which shall be administered to the patient prior to execution of the target-controlled infusion protocol. At the start of execution of the target-controlled infusion protocol, hence, it is taken into account whether previously an induction dose has been administered to the patient according to user-specified settings, i.e., outside of the regime of the automatic control in the context of the target-controlled infusion protocol. When executing the target-controlled infusion protocol, hence, the control does not start fresh by assuming a 0 drug concentration within the patient, but the target-controlled infusion protocol takes into account that an induction dose has been administered to the patient prior to the start of execution of the target-controlled infusion protocol.

Because a user may administer a manual induction dose to the patient prior to executing the automatic control according to the target-controlled infusion protocol, and because the manual induction dose is taken into account when starting the automatic control, a user is enabled to start an administration operation, for example an infusion operation, by giving a manual induction dose. As the user, according to this experience from manual, non-automatic systems, may be familiar with conducting an administration operation by employing induction doses according to manual user specifications, this may increase acceptance for an automatic control employing a target-controlled infusion protocol, in that the process is started with a familiar induction dose and only subsequently switches to an automatic control of the target-controlled infusion protocol. The user hence may administer or specify the manual induction dose according to his experience, and may then cause the control to switch to the automatic execution of the target-controlled infusion protocol.

There are different possible options by which a user-specified induction dose may be administered to the patient prior to execution of the target-controlled infusion protocol.

In a first option, a user may manually administer an induction dose in the shape of a bolus to the patient prior to execution of the target-controlled infusion protocol. When starting the execution of the target-controlled infusion protocol, information relating to the prior induction dose is taken into account in order to compute a drug concentration at the start of execution of the target-controlled infusion protocol and to carry out the target-controlled infusion protocol based on this computed drug concentration.

In a second option, a user may specify an induction dose which shall be administered to a patient prior to executing the target-controlled infusion protocol. Hence, a bolus according to user specifications is administered to the patient prior to executing the target-controlled infusion protocol, wherein at the start of executing the target-controlled infusion protocol a drug concentration which is computed based on the user-specified induction dose is taken into account for carrying out the target-controlled infusion protocol.

In a conventional target-controlled infusion protocol, at the start of execution of the target-controlled infusion protocol a bolus is administered to the patient, wherein the induction dose to be given within the bolus is automatically computed by the target-controlled infusion protocol. In contrast thereto, in the second option the initial bolus is not automatically computed, but is administered according to user specifications, such that the initial bolus prior to starting the actual target-controlled infusion is set according to user specifications.

In a third option, a user may specify an induction dose according to a common dose rate protocol (for example in mg/kg/h) or a common flow rate protocol (for example in ml/h). The induction dose according to the common dose rate protocol or flow rate protocol, which may take into account for example a dose information and a duration information indicating a time span over which the dose shall be administered, shall be administered to the patient prior to executing the target-controlled infusion protocol, such that the administration starts with a user-specified process, and only upon terminating the user-specified process it is switched to the target-controlled infusion.

The control device, in one embodiment, is a separate device to the infusion device. The control device herein may serve to control operation of one or multiple infusion devices.

In another embodiment, the control device may be incorporated into an infusion device, such that the control device may be implemented by the infusion device and hence does not necessarily form a separate entity with respect to the at least one infusion device.

Further, the control device may be embodied by multiple entities. For example, a portion of the control device may be implemented by the infusion device, i.e., a processor off the infusion device, wherein another portion of the control device may be implemented by a device separate to the infusion device.

In one embodiment, the control device is configured to cause a display prompt requesting a user input to specify information concerning the infusion dose administered to the patient prior to the display prompt or to be administered to the patient after the display prompt. In the display prompt, the user may have to enter information concerning the induction dose in order to specify the induction dose. The induction dose herein may have already been administered to the patient, or may have to be administered to the patient prior to executing the target-controlled infusion protocol. The user enters hence a dose information, and based on the dose information the control device computes and considers the value for the drug concentration at the start of execution of the target-controlled infusion protocol.

The induction dose may be specified for example by a dose information and a time information. In particular, a user may input a dose information specifying the mass of the dose and a time information specifying at which time the dose has been administered to the user prior to the display prompt. When starting the target-controlled infusion protocol, the dose information and the time of administration may be taken into account in order to compute the drug concentration within the patient at the start of executing the target-controlled infusion protocol.

In the second option in which the induction dose shall be given according to the user specifications prior to execution of the target-controlled infusion protocol, only a dose information may be entered, specifying the bolus to be given, wherein the induction dose is administered to the patient, and subsequently it is switched to the automatic control of the target-controlled infusion protocol.

In another embodiment, the information to be entered by the user may include a combination of at least two of a dose information, a duration information specifying a duration of administration and a flow rate information. According to the third option, prior to executing the target-controlled infusion protocol to establish an automatic control, a dose rate protocol (or a flow rate protocol) shall be executed in order to administer a defined induction dose to the patient according to the dose rate protocol (or the flow rate protocol). For the flow rate protocol, for example the induction dose and the duration may have to be entered by the user, wherein in the dose rate protocol the required flow rate may be computed automatically and, subsequently, the induction dose is administered to the patient according to the specifications within the dose rate protocol.

The display prompt may be displayed to a user in a workflow when programming the system for executing a target-controlled infusion. The display prompt herein may be displayed to the user during the course of programming the system, wherein at conclusion of the programming the administration operation is started.

In one embodiment, the control device is configured to control operation of the at least one infusion device selectively in a manual induction mode and in an automatic induction mode. In the manual induction mode the control device is configured to calculate, based on the user-specified induction dose, the drug concentration value within the patient using the mathematical model and to consider the drug concentration at the start of execution of the target-controlled infusion protocol. In the automatic induction mode the control device assumes a 0 drug concentration in the patient at the start of execution of the target-controlled infusion protocol.

The system hence may be operated in a manual induction mode and in an automatic induction mode. In the manual induction mode a user-specified induction dose is administered to the patient prior to execution of the target-controlled infusion protocol. Hence, in the manual induction mode a user initially specifies an induction dose which already has been administered to the patient or which shall be administered to the patient prior to execution of the target-controlled infusion operation. When starting the target-controlled infusion protocol it is taken into account what concentration within the patient arises due to the manual induction dose, such that the automatic control based on the target-controlled infusion protocol takes into account the prior administration of the manual induction dose. In the automatic induction mode, in contrast, the administration takes place automatically without a user specification for an initial induction dose. Hence, for the automatic control it is assumed that no manual induction dose has been administered to the patient prior to starting the target-controlled infusion protocol, such that a zero drug concentration is assumed within the patient. The automatic induction mode hence corresponds to a conventional mode of a target-controlled infusion.

In one embodiment, within a workflow for programming an administration operation a user may be displayed a display prompt, the display prompt requesting the user to select the manual induction mode or the automatic induction mode. The user, during the programming, hence selects whether a target-controlled infusion shall be carried out by applying a user-specified induction dose, or by applying an automatic induction dose.

In one embodiment, the control device is configured to cause a display prompt displaying the value of the drug concentration at the start of execution of the target-controlled infusion protocol. On a display the user hence is presented with concentration information resulting from the user-specified induction dose administered prior to execution of the target-controlled infusion protocol. The user hence is informed about a drug concentration within the patient resulting from the user-specified induction dose.

In one embodiment, the control device is configured to assume a target concentration at the start of execution of the target-controlled infusion protocol corresponding to the value of the drug concentration. Hence, at the beginning of the automatic control according to the target-controlled infusion protocol the target concentration is set according to the computed drug concentration as a result of the user-specified induction dose.

Whereas at the beginning of executing the target-controlled infusion protocol the target concentration may be set according to the computed concentration, it may be desirous to subsequently set the target concentration to another value in order to achieve a desired effect at an effect site. Hence, the target concentration, after the initial setting according to the computed drug concentration as a result from the user-specified induction dose, may be varied in order to adjust the target concentration towards a more realistic target concentration. For example, the target concentration may be ramped up by linearly increasing the target concentration within a specified time span after the start of execution of the target-controlled infusion protocol.

The time span herein may for example be user-specified during the programming in that the user enters within what time an actual target concentration shall be reached within the patient.

In one embodiment, the control device is configured to control operation of the at least one infusion device in order to establish a drug concentration at an effect site or at a plasma site within the patient based on the target concentration. The effective site may for example correspond to the patient's brain, for example to achieve a desired anesthetic effect within the patient. The plasma site in particular may correspond to a patient's blood compartment, such that by means of the control a specified drug concentration in the patient's blood is established.

The mathematical model in particular may be a pharmacokinetic/pharmacodynamic model which models the drug distribution of a drug administered to a patient. Within the pharmacokinetic/pharmacodynamic model the drug concentration is modeled in different body compartments of a patient, in particular a plasma compartment, a brain compartment, a rapid equilibrating compartment (representative e.g. of muscle and inner organ tissue) and a slow equilibrating compartment (e.g. fat, bone tissue). The model herein may self-adjust during execution of the target-controlled infusion protocol in dependence on measurement values as obtained during execution, such that the model is individualized during execution and hence reflects patient-specific conditions as experienced during the target-controlled infusion operation. For this, in particular the control device may be configured to adjust at least a subgroup of a multiplicity of parameters of the mathematical model during execution of the target-controlled infusion protocol according to measurement values relating to a drug concentration distribution in the patient.

In another aspect, a method for controlling a target-controlled infusion for administering a drug to a patient comprises: controlling, using a control device, operation of at least one infusion device in order to establish a drug concentration within the patient based on a target concentration by executing a target-controlled infusion protocol using a mathematical model modelling a drug distribution in the patient's body for controlling operation of the at least one infusion device; and calculating, by the control device and based on a user-specified induction dose, a value of a drug concentration within the patient using said mathematical model and considering said value of the drug concentration at the start of execution of the said target-controlled infusion protocol.

The advantages and advantageous embodiments as described above for the system equally apply also to the method, such that it shall be referred to the above in this respect.

Within the method, a user may be prompted, prior to the calculating, in a display prompt to specify information concerning the induction dose administered to the patient prior to the prompting or to be administered to the patient after the prompting. A user hence enters information concerning an induction dose which already has been (manually) administered to the patient or which shall be administered to the patient prior to executing the target-controlled infusion protocol and hence prior to an automatic control.

The idea underlying the invention shall subsequently be described in more detail by referring to the embodiments shown in the figures. Herein:

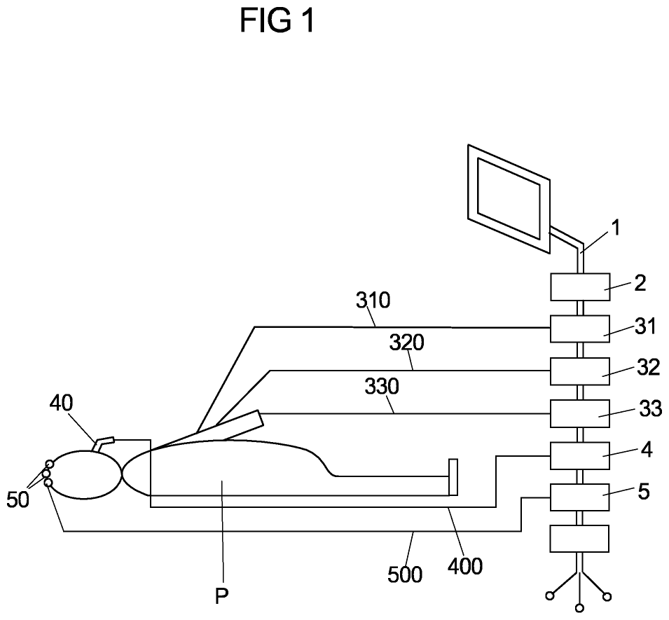

FIG. 1 shows a schematic view of a setup of system for performing a target-controlled infusion (TCI);

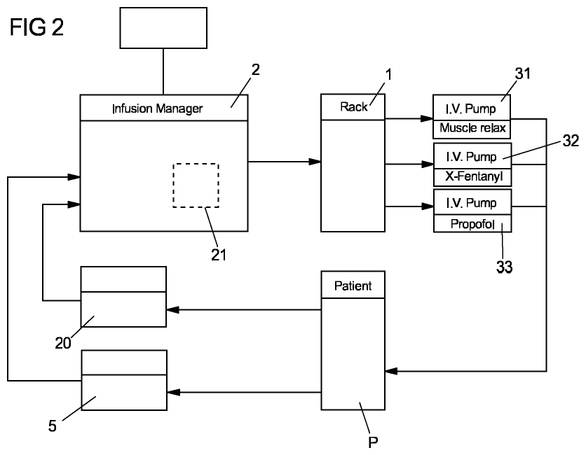

FIG. 2 shows a functional diagram of the setup of FIG. 1;

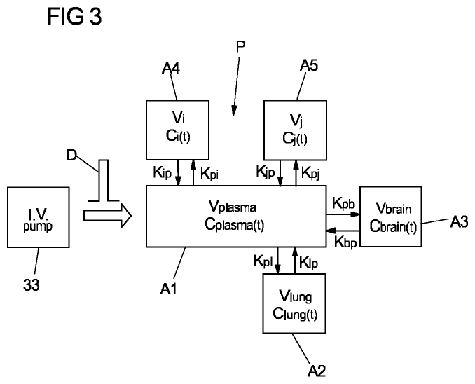

FIG. 3 shows a functional diagram of a model for modelling the distribution of a drug dosage in a patient's body;

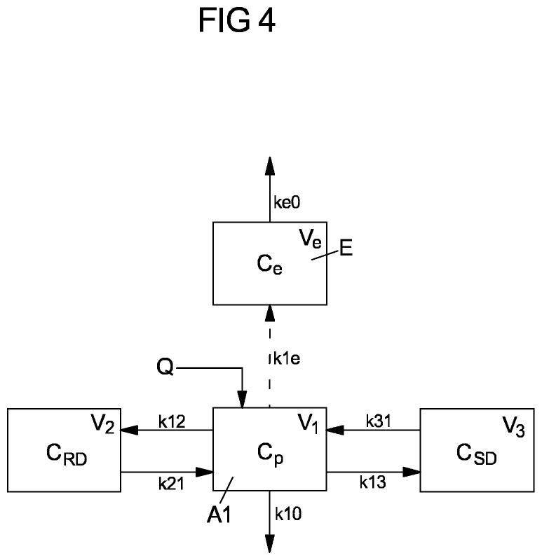

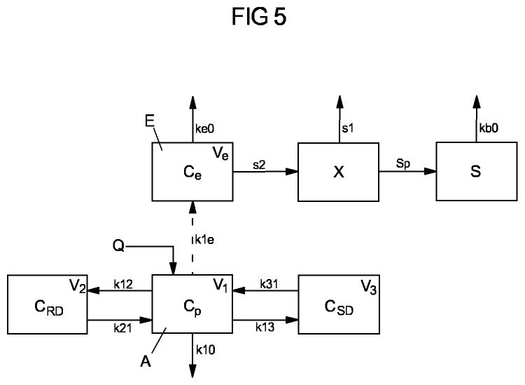

FIG. 4 shows a schematic diagram of a PK/PD model;

FIG. 5 shows a schematic diagram of another PK/PD model;

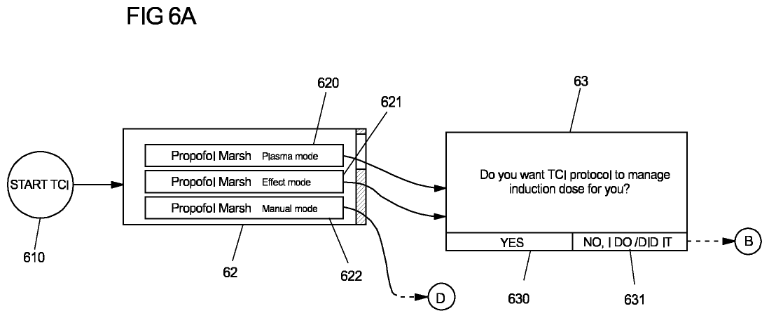

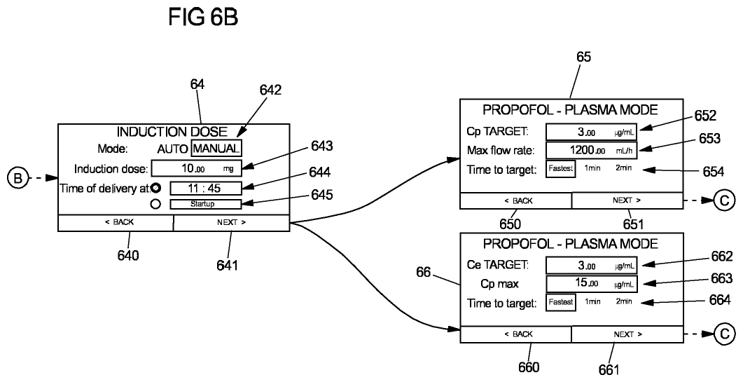

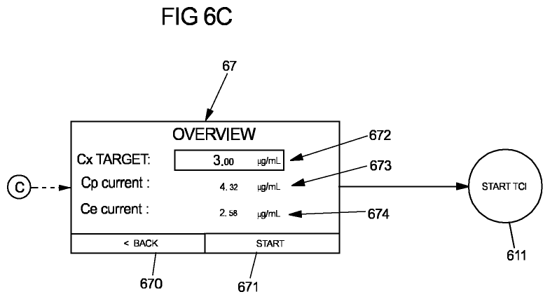

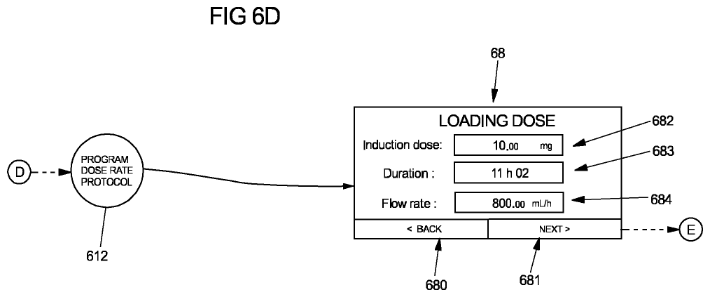

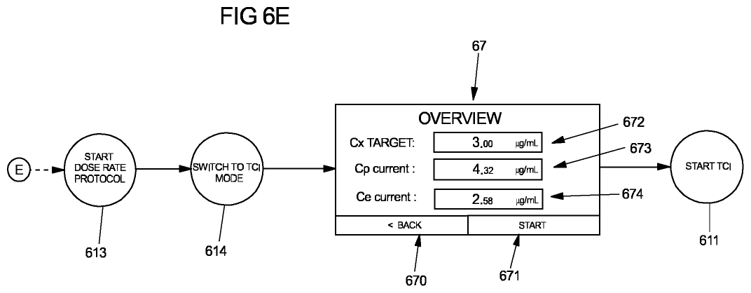

FIG. 6A shows a view of a workflow for programming an administration operation by employing a target-controlled infusion;

FIG. 6B shows a view of the workflow of FIG. 6A, continued;

FIG. 6C shows a view of the workflow according to FIGS. 6A and 6B, continued;

FIG. 6D shows a view of the workflow of FIGS. 6A to 6C, when choosing another mode;

FIG. 6E shows a view of the workflow of FIG. 6D, continued;

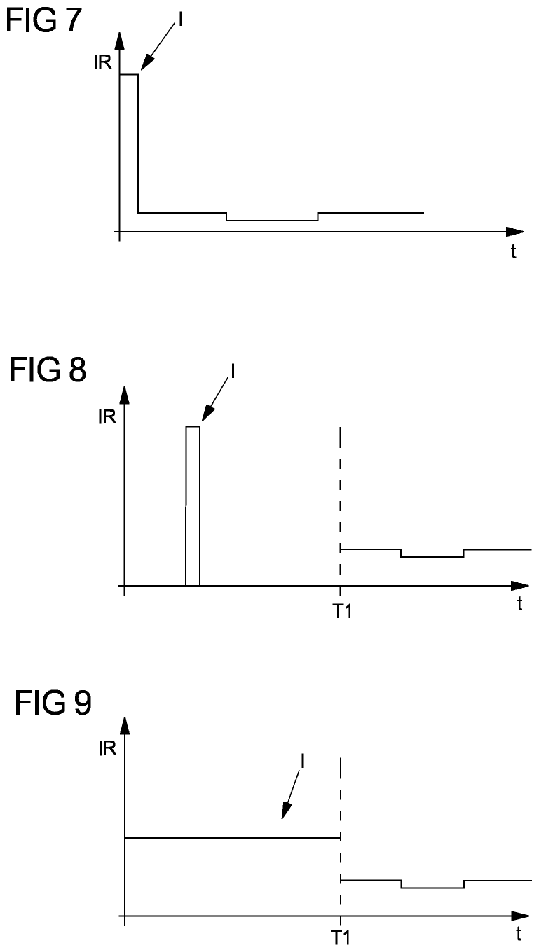

FIG. 7 shows a view of an infusion rate over time, in a conventional, automatic target-controlled infusion scheme;

FIG. 8 shows a view of a scheme in which a manual induction dose is administered prior to starting a target-controlled infusion;

FIG. 9 shows a view of a scheme in which an induction dose is administered to the patient prior to starting a target-controlled infusion;

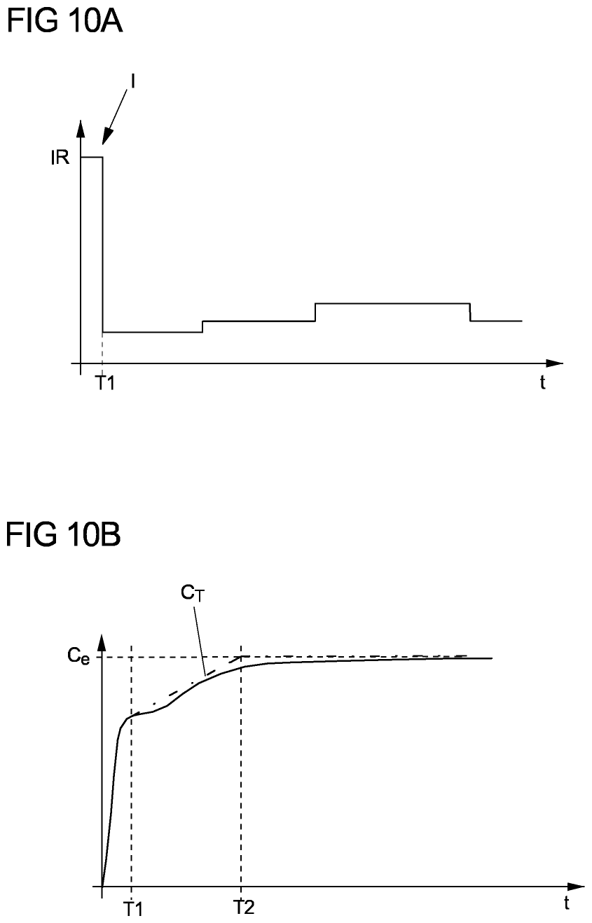

FIG. 10A shows a view of yet another scheme in which an induction dose according to user specifications is administered to the patient prior to starting a target-controlled infusion; and

FIG. 10B shows a view of a drug concentration within a patient at an effect site, corresponding to the infusion rate profile of FIG. 10A.

Subsequently, a system and method for administering one or multiple drugs to a patient in a target-controlled infusion (TCI) procedure, e.g. an anesthetic procedure, shall be described in certain embodiments. The embodiments described herein shall not be construed as limiting for the scope of the invention.

Like reference numerals are used throughout the figures as appropriate.

FIG. 1 shows a schematic drawing of a setup as it generally is used for example in an anesthesia procedure for administering anesthetic drugs, such as analgesic agents or hypnotic agents, e.g. propofol and/or remifentanil, to a patient P. In this setup multiple devices are arranged on a rack 1 and are connected via different lines to the patient P.

In particular, infusion devices 31, 32, 33 such as infusion pumps, in particular syringe pumps or volumetric pumps, are connected to the patient P and serve to intravenously inject, via lines 310, 320, 330, different drugs such as propofol, remifentanil and/or a muscle relaxant drug to the patient P in order to achieve a desired anesthetic effect. The lines 310, 320, 330 are for example connected to a single port providing access to the venous system of the patient P such that via the lines 310, 320, 330 the respective drugs can be injected into the patient's venous system.

The rack 1 furthermore may hold a ventilation device 4 for providing an artificial respiration to the patient P e.g. while the patient P is under anesthesia. The ventilation device 4 is connected via a line 400 to a mouth piece 40 such that it is in connection with the respiratory system of the patient P.

The rack 1 also holds a bio-signal monitor 5, for example an EEG monitor which is connected via a line or a bundle of lines 500 to electrodes 50 attached to the patient's head for monitoring the patient's brain activity e.g. during an anesthesia procedure.

In addition, a control device 2 is held by the rack 1 which serves to control the infusion operation of one or multiple of the infusion devices 31, 32, 33 such that infusion devices 31, 32, 33 inject drugs to the patient P in a controlled fashion to obtain a desired effect, e.g. an anesthetic effect. This shall be explained in more detail below.

It is to be noted herein that the control device 2 may also be incorporated into an infusion device 31, 32, 33, such that the control device 2 may be implemented by the infusion device 31, 32, 33.

Additional measurement devices may be used, e.g. for measuring the concentration of one or multiple drugs for example in the breath of the patient P or to measure information relating to and allowing to determine e.g. a bi-spectral index. A measurement device may for example be constituted by a so called IMS monitor for measuring a drug concentration in the patient's breath by means of the so called Ion Mobility Spectrometry. Other sensor technologies may also be used.

FIG. 2 shows a functional diagram of a control loop for controlling the infusion operation of infusion devices 31, 32, 33 during an infusion operation. The control loop herein may in principle be set up as a closed-loop in which the operation of the infusion devices 31, 32, 33 is automatically controlled without user interaction. Alternatively, the system is set up as an advisory (open-loop) system in which at certain points of time, in particular prior to administering a drug dosage to a patient, a user interaction is required in order to manually confirm the operation.

The control device 2, also denoted as “infusion manager”, is connected to the rack 1 which serves as a communication link to the infusion devices 31, 32, 33 also attached to the rack 1. The control device 2 outputs control signals to control the operation of the infusion devices 31, 32, 33, which according to the received control signals inject defined dosages of drugs to the patient P.

By means of the bio-signal monitor 5, e.g. in the shape of an EEG monitor, for example an EEG reading of the patient P is taken, and by another measurement device 20 a concentration of one or multiple drugs in the patient's P breath is measured. The measured data are fed back to the control device 2, which correspondingly adjusts its control operation and outputs modified control signals to the infusion devices 31, 32, 33 to achieve a desired anesthetic effect.

The control device 2 uses, for controlling the infusion operation of one or multiple infusion devices 31, 32, 33, a pharmacokinetic-pharmacodynamic (PK/PD) model, which is a pharmacological model for modelling processes acting on a drug in the patient's P body. Such processes include the resorption, the distribution, the biochemical metabolism and the excretion of the drug in the patient's P body (denoted as pharmacokinetics) as well as the effects of a drug in an organism (denoted as pharmacodynamics). Preferably, a physiological PK/PD model with N compartments is used for which the transfer rate coefficients have been experimentally measured beforehand (for example in a proband study) and are hence known.

A schematic functional drawing of the setup of such a PK/PD model p is shown in FIG. 3. The PK/PD model p logically divides the patient P into different compartments A1-A5, for example a plasma compartment A1 corresponding to the patient's P blood stream, a lung compartment A2 corresponding to the patient's P lung, a brain compartment A3 corresponding to the patient's P brain and other compartments A4, A5 corresponding, for example, to muscular tissue or fat and connective tissue. The PK/PD model p takes into account the volume V_{Lung}, Vₚₗₐₛₘₐ, V_{brain}, Vᵢ, Vⱼ of the different compartments A1-A5 as well as transfer rate constants K_{PL}, K_{LP}, K_{BP}, K_{PB}, K_{IP}, K_{PI}, K_{JP}, K_{PJ} indicating the transfer rates between the plasma compartment A1 and the other compartments A2-A5, assuming that a drug dosage D by means on an infusion device 31-33 is injected into the plasma compartment A1 and the plasma compartment A1 links the other compartments A2-A5 such that an exchange between the other compartments A2-A5 always takes place via the plasma compartment A1. The PK/PD model p serves to predict the concentrations C_{lung}, Cₚₗₐₛₘₐ, C_{brain}, Cᵢ, Cⱼ of the injected drug in the different compartments A1-A5 as a function of time.

FIG. 4 illustrates, in a schematic diagram, an example of a PK/PD model which comprises a central plasma compartment A1 exhibiting a drug concentration Cₚ, a rapid equilibrating compartment exhibiting a drug concentration C_{RD}, a slow equilibrating compartment exhibiting a drug concentration C_{SD}, an dan effect compartment E comprising an effect compartment concentration Cₑ of the drug. Herein,
Q represents an administered drug,
kₑ₀ defines the proportional change in each unit of time of the concentration gradient between the plasma and effect-site,
k₁ₑ describes an elimination constant for redistribution of the drug from the effect compartment E to the plasma compartment A1,
k₁₂ is an elimination constant describing the distribution of the volume V1 in direction of volume V2,
k₂₁, is an elimination constant describing the distribution of the volume V2 in direction of volume V1,
k₁₃ is an elimination constant describing the distribution of the volume V1 in direction of volume V3,
k₃₁ is an elimination constant describing the distribution of the volume V3 in direction of volume V1,
k₁₀ represents the elimination constant of the applied drug from the body.

FIG. 4 herein visualizes a so called Schnider model. This assumes that after intravenous injection a drug Q is rapidly distributed in the circulation of the central plasma compartment A1 and quickly reaches well perfused tissues, such that a tissue-specific redistribution in various other compartments such as muscle or fat tissue occurs. At the same time the body eliminates the applied substance from the plasma compartment A1 with a certain elimination rate. For the pharmacokinetic characterization e.g. of lipophilic anesthetics, a 3-compartment model has been established that comprises a plasma compartment A1 (heart, lung, kidney, brain), a rapid equilibrating compartment exhibiting a concentration C_{RD} (muscles, inner organs), and a slow equilibrating compartment exhibiting a concentration C_{SD} (fat, bone, the so-called “deep” compartment). The concentration-time curve of a drug is characterized by the distribution volume of a specific compartment and the clearance (which is the plasma volume, from which the drug is eliminated per time unit): V1 denotes the volume of the plasma compartment A1, V2 is the volume of a well-perfused tissue C_{RD} and V3 is the volume of a less perfused compartment, associated with a concentration C_{SD}. The clearance of a substance from the various compartments can be described by elimination constants. The elimination constant k₁₂ for example describes the distribution from the volume V1 towards the volume V2, and k₂₁, describes the distribution in the opposite direction. An applied substance is eliminated by this model with the constant k₁₀ from the body. After reaching an equilibrium (“steady state”) between the individual compartments, the elimination rate determines the amount of substance that must be supplied to maintain equilibrium.

To assess the clinical effect (the so-called pharmacodynamics) of a drug at the target site, dose-response curves are generally used. Such curves, which are typically of a sigmoidal shape, describe the association between the drug concentration and a particular clinical effect. Knowing the dose-response relationship, a putative drug concentration at the site of action, the effect compartment E, can be calculated.

FIG. 5 shows a schematic diagram of another example of a PK/PD model, as it for example is described in WO 2017/190966 A1. In comparison to the model of FIG. 4, the model additionally comprises a remote compartment X and a BIS sensor compartment S, wherein
s1 and s2 represent constant transfer rate parameters between the remote compartment X and the effect compartment E,
S_{P} represents a transfer rate coefficient between the remote compartment X and the BIS sensor S, and
k_{b0} represents the decay rate of the BIS index.

Clinically, S_{P} can be regarded as a sensitivity value. The higher the value of S_{P}, the faster the drug's effect is achieved. High values of S_{P} further lead to a small delay and a high responsiveness of the system.

The remote compartment X describes the delay between the drug's concentration in the effect-site compartment and its actual impact on the BIS value.

TCI models, e.g. for propofol, are known in the art. Recently introduced open-target-controlled infusion systems can be programmed with any pharmacokinetic model, and allow either plasma- or effect-site targeting. With effect-site targeting the goal is to achieve a user-defined target effect-site concentration as rapidly as possible, by manipulating the plasma concentration around the target. Currently systems are for example pre-programmed with a Marsh model (B. Marsh et al., “Pharmacokinetic model driven infusion of propofol in children” Br J Anaesth, 1991; 67, pages 41-48) or a Schnider model (Thomas W. Schnider et al., “The influence of method of administration and covariates on the pharmacokinetics of propofol in adult volunteers”, Anesthesiology, 1998, 88(5) pages 1170-82).

The PK/PD model as shown in FIG. 5 can mathematically be described by the following set of (differential) equations.

Namely, the S compartment is described according to:

$$\dot{S} = \frac{{s}_{p} X}{{α}_{M} + X} - {k}_{b 0} S + OF \qquad ( Equation 1 )$$

wherein
s_{P} represents the drug sensitivity of the patient;
α_{M} represents the saturation parameter of the velocity of effect of a drug, e.g. an anesthetic agent such as propofol (i.e. the saturation of the drug receptors);
k_{b0} represents the decay rate of the BIS index;
OF represents the offset that can remain when no more drug is present in the patient body;
X represents a remote compartment; and
S represents a sensor value of a BIS sensor.

The X compartment is described by:

$$\dot{X} = {s}_{2} {C}_{e} - {s}_{1} X . \qquad ( Equation 2 )$$

wherein
s₁ and s₂ represent constant transfer rate parameters between the remote compartment and the effect parameters;
Cₑ represents the effect compartment concentration; and
X represents a remote compartment.

The rapid equilibrating compartment C_{RD} is described by:

$${\dot{C}}_{RD} = - {k}_{21} {C}_{RD} + {k}_{12} {C}_{p} \qquad ( Equation 3 )$$

wherein
k₁₂ is an elimination constant describing the drug distribution from the plasma compartment A1 in direction of rapid equilibrating compartment C_{RD},
k₂₁ is an elimination constant describing the drug distribution from rapid equilibrating compartment C_{RD} in direction of plasma compartment A1,
C_{RD} represents the concentration in the rapid equilibrating compartment, and
Cₚ represents the drug concentration in the plasma (blood) compartment.

The slow equilibrating compartment C_{SD} is described by:

$${\dot{C}}_{SD} = {k}_{31} {C}_{SD} + {k}_{13} {C}_{p} \qquad ( Equation 4 )$$

wherein
k₁₃ is an elimination constant describing the drug distribution from plasma compartment A1 in direction of slow equilibrating compartment C_{SD},
k₃₁ is an elimination constant describing the drug distribution from slow equilibrating compartment C_{SD} in direction of plasma compartment A1,
C_{SD} represents the concentration in the slow equilibrating compartment; and
Cₚ represents the drug concentration in the plasma (blood) compartment.

The effect compartment concentration Cₑ is described by:

$${\dot{C}}_{e} = - {k}_{e 0} {C}_{e} + {k}_{1 e} {C}_{p} \qquad ( Equation 5 )$$

wherein
kₑ₀ defines a decay rate;
k₁ₑ describes a “virtual” constant rate transfer from plasma compartment A1 and the effect compartment E; and
Cₑ represents the effect comportment concentration.

The blood concentration Cₚ is described by:

$${\dot{C}}_{p} = - ( {k}_{10} + {k}_{12} + {k}_{13} ) {C}_{p} + {k}_{21} {C}_{RD} + {k}_{31} {C}_{SD} \qquad ( Equation 6 )$$

wherein
k₁₀ represents the elimination constant of an applied drug from the body,
k₁₂ is an elimination constant describing the drug distribution from the plasma compartment A1 in direction of rapid equilibrating compartment C_{RD},
k₂₁ is an elimination constant describing the drug distribution from rapid equilibrating compartment C_{RD} in direction of plasma compartment A1,
k₁₃ is an elimination constant describing the drug distribution from plasma compartment A1 in direction of slow equilibrating compartment C_{SD},
k₃₁ is an elimination constant describing the drug distribution from slow equilibrating compartment C_{SD} in direction of plasma compartment A1,
C_{RD} represents a rapid equilibrating compartment;
C_{SD} represents a slow equilibrating compartment; and
Cₚ represents the drug concentration in the plasma (blood) compartment.

Generally, the mathematical model, e.g. a PK/PD model as described above, during execution of an infusion operation is used to model the drug concentrations in certain body compartments of the patient, such that information about the drug distribution during the infusion operation is available for controlling the infusion operation using one or multiple infusion devices. The control herein is such that e.g. at the effect site, for example in the patient's brain, a drug concentration is established which is at or close to a desired target concentration, wherein for this the control device 2 (FIGS. 1 and 2) controls infusion devices 31-33 such that a drug is infused to reach and maintain a drug concentration at the relevant site at or close to the desired target concentration.

During execution of the infusion operation, the mathematical model may be tuned according to measurement values as obtained for example by a bio-signal monitor or from a sensor for measuring a drug concentration in the exhaled breath of a patient or the like. Using measurement information the mathematical model may be tuned according to actual concentration information by adjusting parameters of the model, for example transfer rate parameters or the like, such that the model correctly reflects the measured information and hence reliably predicts the drug concentrations in the different body compartments.

Referring now to FIG. 7, in a conventional target-controlled infusion an infusion rate IR is controlled such that an initial induction dose I is administered to the patient in order to establish a drug concentration within the patient which in a fast manner converges towards a desired target concentration. Subsequent to the initial induction dose I the infusion rate IR is set such that the concentration within the patient, for example at an effect site or in a plasma site, is maintained at or at least close to the desired target concentration.

Control herein is carried out by employing a target-controlled infusion protocol employing a mathematical model as described above and by computing concentrations in different compartments within the patient in order to determine a drug distribution within the patient using the mathematical model.

In a conventional target-controlled infusion the initial induction dose I is determined automatically in dependence on the desired target concentration. The initial induction dose hence is not user-specified, but computed by the system, in accordance with a setting for a target concentration at an effect site or a plasma site.

It herein is proposed to deviate from an automatic control as conventionally applied, but to use a control in which a user initially is allowed to specify an induction dose I, wherein a subsequent automatic control based on a target-controlled infusion protocol takes place by taking into account the prior user-specified induction dose I.

The user hence is allowed to specify an induction dose, which may be more familiar with a user, as it resembles a conventional, manual administration based on specifying e.g. an infusion dose and a duration of administration. A user, who is not familiar for example with setting a target concentration at an effect site or a plasma site, but is more familiar with specifying an infusion dose, may gain an easier access to an automatic control employing a target-controlled infusion.

Referring now to FIGS. 6A to 6E, in a workflow for programming an infusion operation a user may be requested to enter information to specify an initial induction dose, which has already been administered to a patient or which shall be administered to a patient prior to executing a target-controlled infusion protocol.

Referring now to FIG. 6A, in the workflow the programming sequence is started at execution stage 610. In a first display prompt 62 a user, on a display, is prompted with input fields 620, 621, 622, in which the user may select a particular mode for carrying out the administration. Depending on his choice, the user then is displayed another display prompt 63, in which the user may enter whether he wishes to conduct an automatic control using a regular target-controlled infusion protocol (input field 630) or whether an induction dose has already been administered or shall be administered to the patient according to user-specified settings (input field 631).

If the user selects the input field 630, for example by tapping on the input field 630 on a touch sensitive display, an automatic control according to a target-controlled infusion operation is initiated. This represents an automatic induction mode in which an initial induction dose is determined automatically by the system.

If, instead, the user selects the input field 631, the user is displayed the display prompt 64 as shown in FIG. 6B and hence is requested to enter information concerning an induction dose I which already has been administered to the patient or which shall be administered to the patient prior to the automatic control.

For example, if the user in the input field 642 selects “Manual”, in the input field 643 enters an induction dose value (in the example “10 mg”), and in the input field 644 enters a time at which the induction dose has been administered (in the example “11:45”), and subsequently selects the input field 641 to continue, the user is displayed one of the display prompts 65, 66, dependent on the choice of mode in the display prompt 62 according to FIG. 6A.

Namely, if the user in the display prompt 62 has selected “Plasma mode” (input field 620), the user is displayed the display prompt 65. If instead the user has, in the display prompt 62, selected “Effect mode” (input field 621), the user is displayed the display prompt 66.

In the plasma mode a drug concentration, during the target-controlled infusion, is set in the plasma compartment according to a target concentration. In the display prompt 65, accordingly, a target concentration may be set for the plasma compartment (input field 652). In addition, a “Maximum flow rate” (input field 653) and a “Time to target” (input field 654) may be set.

In the effect mode, in contrast, a drug concentration is set at the effect site according to a specified target concentration. In the display prompt 66, accordingly, a target concentration may be set for the effect site compartment (input field 662). In addition, a “Maximum flow rate” (input field 663) and a “Time to target” (input field 664) may be set.

By selecting the input field 651 respectively 661, the user continues and is displayed the display prompt 67 as shown in FIG. 6C. In particular, the user is informed about the set target concentration (field 672) and is informed about the current concentration values Cₚ, Cₑ in the plasma compartment respectively the effect site compartment (fields 673, 674).

By selecting the input field 671 the user may start the target-controlled infusion operation and hence an automatic control of the administration.

In the shown example a user, in the display prompt 64 according to FIG. 6B, specifies an induction dose which has been administered to the patient prior to the start of execution of the target-controlled infusion. The induction dose may have been administered using an infusion device 31, 32, 33, or may have been administered manually by means of a suitable access port in an infusion set connected to the patient.

According to the specified induction dose of the display prompt 64, the system computes initial concentration values Cₚ, Cₑ, which result from the induction dose. The system herein takes into account the induction dose information (input field 643) as well as the time of administration (input field 644), and based on the dose and the time of administration computes, e.g. by employing a mathematical model as described above, the concentration values Cₚ, Cₑ as displayed in the display prompt 67 of FIG. 6C. When starting the target-controlled infusion operation (execution stage 611), the drug distribution within the patient resulting from the prior induction dose is taken into account, such that the target-controlled infusion starts from the initial induction dose as given by the user and as specified to the system in the display prompt 64.

In the display prompt 65, 66 a “Time to target” may be selected in input field 654, 664. If “Fastest” is selected, the system will control the infusion rate such that the target concentration is reached as fast as possible. If “1 min” or “2 min” is selected, it is assumed that the target concentration shall be reached only within a time span of 1 minute respectively 2 minutes. In this case an initial target concentration may be set to the computed concentration at the relevant site (plasma site, effect site) and may be ramped up towards the target concentration as specified in input field 652, 662 such that the target concentration is linearly increased from the initial, computed concentration (as displayed in fields 673, 674 in the display prompt 67 of FIG. 6C) towards the specified target concentration of input field 652, 662.

In the display prompt 64, the user may choose, by activating input field 645 instead of entering a “Time of delivery” into input field 644, that the induction dose shall be administered at the start of the administration operation, prior to the actual control based on the target-controlled infusion protocol.

The system herein may react to an input in the display prompt 64 in different ways:

If the induction dose specified in the display prompt 64, by entering the induction dose in input field 643 and by selecting “Startup” in input field 645, is smaller than an induction dose which is computed by the system as required to reach a predefined target concentration, the system may
(a) use the computed induction dose rather than the user-specified induction dose, in particular if in input field 654, 664 the “Time to target” option “fastest” is selected, or
(b) may use the user-specified induction dose, may set the initial target concentration to the computed concentration as a result of the user-specified induction dose, and may ramp up the target concentration from the initial target concentration to the specified target concentration of input field 652, 662 according to a specified time span, in particular if in input field 654, 664 a 1-minute or 2-minute time span is selected.

In contrast, if the induction dose as specified in the display prompt 64 is larger than a computed induction dose, the system may use the user-specified induction dose, for any “Time to target” option in input field 654, 664 of the display prompt 65, 66.

Referring now again to FIG. 6A, in the display prompt 62 also the input field 622 may be selected and hence a so-called “Manual mode” may be entered. If this input field 622 is selected, the workflow continues as shown in FIG. 6D by entering a programming routine for a dose rate protocol (execution stage 612).

Thereupon, a display prompt 68 is displayed to the user, by which the user may specify an induction dose (input field 682), a duration (input field 683) and a flow rate (input field 684). By selecting the input field 681 it is continued to execution stage 613, which initiates the administration according to the dose rate protocol as specified in display prompt 68 (FIG. 6E).

At the execution stage 614, after concluding the administration according to the dose rate protocol in execution stage 613, it is switched to the target-controlled infusion, and in display prompt 67 a target concentration may be adjusted (input field 672) and the current concentrations Cₚ, Cₑ in the plasma compartment and at the effect site are displayed (fields 673, 674).

By pressing the field 671 the automatic control according to the target-controlled infusion may then be started (execution stage 611).

In any of the display prompts 64-68 the user may revert to the previous display prompt 63-67 by selecting the “Back” field 640, 650, 660, 670, 680.

In the different options of the workflow as shown in FIGS. 6A to 6E, in any case a user-specified induction dose is administered prior to the start of execution of the target-controlled infusion protocol and is taken into account when starting the target-controlled infusion. The target-controlled infusion hence does not start fresh, but considers a resulting concentration Cₚ, Cₑ (as shown in the display prompt 67 to the user) for the execution of the target-controlled infusion.

Referring now to FIG. 8, in the workflow path of FIGS. 6A to 6C an induction dose I may have been administered manually by a user prior to executing the target-controlled infusion. At the time of programming the target-controlled infusion at time T1, the user, in display prompt 64 of FIG. 6B, enters information about the prior induction dose I, upon which the target-controlled infusion at time T1 is started by taking the prior induction dose I into account and by computing a resulting concentration distribution within the patient at the time T1 of the starting the execution.

Referring now to FIG. 9, in the workflow path of FIGS. 6D and 6E an induction dose I is administered to the patient prior to the automatic target-controlled infusion, the induction dose I being administered according to a loading dose protocol by specifying a desired dose and e.g. a duration over which the dose shall be administered to the patient. At the time T1 the system switches from the loading dose protocol to the target-controlled infusion protocol (execution stage 614 in FIG. 6C), wherein at the start of execution of the target-controlled infusion protocol at the time T1 the concentrations Cₚ, Cₑ resulting from the loading dose protocol prior to time T1 are taken into account for the automatic control.

Herein, for computing the resulting drug concentrations at the time T1, the mathematical model is fed with the loading dose information over time, and from the loading dose over time the resulting drug distribution is computed.

Referring now to FIG. 10A, if in the input prompt 64 of FIG. 6B the option “Startup” of input field 645 is selected (instead of specifying a “Time of delivery” of a prior induction dose), an induction dose according to the user-specified settings is administered prior to the actual start of execution of the target-controlled infusion. This is shown in FIG. 10A, where the induction dose I is administered according to the user-specified settings, and at the time T1 the actual execution of the target-controlled infusion is started.

Herein, if in the display prompt 65, 66 of FIG. 6B a “Time to target” other than “fastest” is selected, the target concentration at the time T1 of starting the target-controlled infusion is set to correspond to the drug concentration at the relevant site as a result of the initial induction dose I.

This is shown in FIG. 10B. At time T1, the target concentration C_{T} is set to the computed drug concentration Cₑ e.g. at the effect site, wherein subsequently the target concentration C_{T} is ramped up between times T1, T2, such that at time T2 the target concentration C_{T} reaches the specified target concentration as set in the input field 652, 662. The time span between times T1, T2 herein equals the time span as specified in the “Time to target” option (input field 654, 664).

The idea of the invention is not limited to the embodiments described above, but may be implemented in a different fashion.

A target-controlled infusion may generally be used for performing an anesthesia operation on a patient, but may also be employed for infusing drugs to a patient to achieve a therapeutic action.

An infusion operation herein may involve one or multiple drugs administered using one or multiple infusion devices.

### LIST OF REFERENCE NUMERALS

1 Rack
2 Control device
20 Measurement device
21 Memory
31, 32, 33 Infusion device
310, 320, 330 Line
4 Ventilation device
40 Mouth piece
400 Line
5 Bio-signal monitor
50 Electrodes
500 Line
610-614 Execution stage
62 Display prompt
620-622 Input fields
63 Display prompt
630, 631 Input fields
64 Display prompt
640-645 Input fields
65 Display prompt
650-654 Input fields
66 Display prompt
660-664 Input fields
67 Display prompt
670-672 Input fields
673, 674 Information fields
68 Display prompt
680-684 Input fields
A1-A5 Compartments
Cₑ Effect site concentration
Cₚ Concentration of plasma compartment
C_{RD} Concentration of rapid equilibrating compartment
C_{SD} Concentration of slow equilibrating compartment
C_{T} Target concentration
D Drug dosage
E Effect site compartment
I Induction dose
IR Infusion rate
k12, k21, k31, k13, k1e, k10 Parameters
kb0 Decay rate
P Patient
Q Drug
S Sensor value
s1, s2 Transfer rate parameter
T1 Time
U Operator (practitioner)
V₁-V₃, Vₑ Volume
X Remote compartment

1. A system for controlling a target-controlled infusion for administering a drug to a patient (P), comprising:
at least one infusion device (31-33) for administering a drug to the patient (P);
a control device (2) which is configured to control operation of the at least one infusion device (31-33) in order to establish a drug concentration within the patient (P) based on a target concentration (CT),
wherein the control device (2) is configured to execute a target-controlled infusion protocol using a mathematical model modelling a drug distribution in the patient's body for controlling operation of the at least one infusion device (31-33);
wherein the control device (2) is configured to calculate, based on a user-specified induction dose (I), a value of a drug concentration within the patient (P) using said mathematical model and to consider said value of the drug concentration at the start of execution of said target-controlled infusion protocol.

2. The system according to claim 1, wherein the control device (2) is configured to cause a display prompt (62-68) requesting a user input to specify information concerning said induction dose (I) administered to the patient (P) prior to the display prompt (62-68) or to be administered to the patient (P) after the display prompt (62-68).

3. The system according to claim 2, wherein said information includes a dose information and a time information to specify a time of administration.

4. The system according to claim 2, wherein said information includes a combination of at least two of a dose information, a duration information specifying a duration of administration and a flow rate information.

5. The system according to claim 1, wherein the control device (2) is configured to control operation of the at least one infusion device (31-33) selectively in a manual induction mode and in an automatic induction mode, wherein in the manual induction mode the control device (2) is configured to calculate, based on said user-specified induction dose (I), said drug concentration value within the patient (P) using said mathematical model and to consider said drug concentration at the start of execution of the said target-controlled infusion protocol, and in the automatic induction mode to assume a zero drug concentration in the patient (P) at the start of execution of the said target-controlled infusion protocol.

6. The system according to claim 5, wherein the control device (2) is configured to cause a display prompt (62-68) requesting a user input to select one of the manual induction mode and the automatic induction mode.

7. The system according to claim 1, wherein the control device (2) is configured to cause a display prompt (67, 77) displaying said value of the drug concentration at the start of execution of the said target-controlled infusion protocol.

8. The system according to claim 1, wherein the control device (2) is configured to assume a target concentration (CT) at the start of execution of the said target-controlled infusion protocol corresponding to said value of the drug concentration.

9. The system according to claim 8, wherein the control device (2) is configured vary the target concentration (CT) after the start of execution of the said target-controlled infusion protocol.

10. The system according to claim 9, wherein the control device (2) is configured to linearly increase the target concentration (CT) within a specified time span after the start of execution of the said target-controlled infusion protocol.

11. The system according to claim 1, wherein the control device (2) is configured to control operation of the at least one infusion device (31-33) in order to establish a drug concentration at an effect site or at a plasma site within the patient (P) based on the target concentration (CT).

12. The system according to claim 1, wherein the mathematical model is a pharmacokinetic/pharmacodynamic model.

13. The system according to claim 1, wherein the mathematical model models a drug concentration in a multiplicity of compartments of the patient (P) during execution of the target-controlled infusion protocol.

14. A method for controlling a target-controlled infusion for administering a drug to a patient (P), comprising:
controlling, using a control device (2), operation of at least one infusion device (31-33) in order to establish a drug concentration within the patient (P) based on a target concentration by executing a target-controlled infusion protocol using a mathematical model modelling a drug distribution in the patient's body for controlling operation of the at least one infusion device (31-33); and
calculating, by the control device (2) and based on a user-specified induction dose (I), a value of a drug concentration within the patient (P) using said mathematical model and considering said value of the drug concentration at the start of execution of the said target-controlled infusion protocol.

15. The method according to claim 14, further comprising prompting a user, prior to said calculating, in a display prompt (62-68) to specify information concerning said induction dose (I) administered to the patient (P) prior to the prompting (62-68) or to be administered to the patient (P) after the prompting.
